(19) United States
(12) Patent Application Publication
(10) Pub. No.: US 2018/0133186 A1
(43) Pub. Date: May 17, 2018

(54) PREVENTATIVE TREATMENT OF ALZHEIMER'S DISEASE

(71) Applicant: Duke University, Durham, NC (US)

(72) Inventor: Carol A. Colton, Durham, NC (US)

(21) Appl. No.: 15/812,686

(22) Filed: Nov. 14, 2017

Related U.S. Application Data
(60) Provisional application No. 62/422,114, filed on Nov. 15, 2016.

(51) Int. Cl. A61K 31/198; A61K 9/00

(52) U.S. Cl. CPC A61K 31/198; A61K 9/0056

(57) ABSTRACT

The present disclosure provides methods and formulations for the treatment of Alzheimer's disease in a subject in need thereof. The methods comprise administering to the subject an ornithine decarboxylase inhibitor such as alpha-difluoromethylornithine or a pharmaceutically acceptable salt in an amount effective to treat the disease.

### CROSS-REFERENCE TO RELATED APPLICATIONS

This application claims priority to U.S. Provisional Application No. 62/422,114 filed on Nov. 15, 2016, the contents of which are incorporated by reference in its entirety.

### STATEMENT REGARDING FEDERALLY SPONSORED RESEARCH

This invention was made with government support under AG045422 awarded by the National Institutes of Health. The government has certain rights in the invention.

### BACKGROUND OF THE INVENTION

The most common neurodegenerative disorder of the brain, Alzheimer's disease, accounts for approximately 70% of all cases of dementia. Alzheimer's disease (AD) is a chronic neurodegenerative disease associated with aging that results in loss of memory and learning ability, loss of executive function and decision making capability, leading to mental and cognitive deterioration and eventually death. Early in the illness the behavioral manifestations of the dementia are often subtle enough to go unnoticed.

In AD, progressive neurodegeneration occurs in multiple areas of the brain, including relatively selective involvement of the nuclei basalis, hippocampus, amygdala, entorhinal cortex, and eventually the high-order association cortex of the temporal, frontal, and parietal regions. The neuronal damage and the attending loss of synaptic density disable several neural systems essential to learning and retrieval of memories.

There are presently no satisfactory treatments to prevent the onset, slow the progression, reverse the pathology or lessen the cognitive damage caused by the disease process (Castellani, R. J.). There continues to be an immediate and critical need for new ways to treat Alzheimer's disease.

### SUMMARY OF THE INVENTION

In one aspect, the disclosure provides a method of treating Alzheimer's disease in a subject in need thereof, the method comprising orally administering said subject a therapeutically effective amount of an ornithine decarboxylase inhibitor or a pharmaceutically acceptable salt thereof.

In another aspect, the disclosure provides an oral dose formulation for treating Alzheimer's disease comprising a dose of an ornithine decarboxylase inhibitor or a pharmaceutically acceptable salt thereof in an amount effective to treat Alzheimer's disease that is both efficacious and demonstrates a low toxicity profile with minimal to no side effects.

In another aspect, the disclosure provides a formulation consisting essentially of an ornithine decarboxylase inhibitor and a pharmaceutically acceptable carrier.

In yet another aspect, the disclosure provides a method of treating dementia associated with Down Syndrome in a subject in need thereof, the method comprising orally administering said subject a therapeutically effective amount of an ornithine decarboxylase inhibitor or a pharmaceutically acceptable salt thereof.

In another aspect, the disclosure provides an oral dose formulation for treating dementia associated with Down Syndrome comprising a dose of an ornithine decarboxylase inhibitor or a pharmaceutically acceptable salt thereof in an amount effective to treat the dementia associated with Down Syndrome that is both efficacious and demonstrates a low toxicity profile with minimal to no side effects.

In another aspect, the disclosure provides a formulation consisting essentially of an ornithine decarboxylase inhibitor and a pharmaceutically acceptable carrier.

In yet another aspect, the disclosure provides a use of alpha-difluoromethylornithine for the making of a medicament for the treatment of Alzheimer's disease or dementia associated with Down Syndrome.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims herein for interpreting the scope of the invention.

### BRIEF DESCRIPTION OF THE DRAWINGS

FIG. 1 shows the experimental schematic for treatment of CVN-AD mice with 0.3 mg/kg or 30 mg/kg DFMO or water only as vehicle control.

FIG. 2 shows the dose-dependent effect of difluoromethylornithine (DFMO) on learning and memory in a mouse model of Alzheimer's disease.

FIGS. 3A-3B show the dose-dependent effect of difluoromethylornithine (DFMO) on Abeta peptides formed from the proteolytic cleavage of amyloid precursor protein in CVN-AD mice, a mouse model of human Alzheimer's disease.

FIG. 4 shows that treatment of CVN-AD mice with DFMO does not alter motor skills or co-ordination as tested by the 3-day rotorod behavioral test compared to CVN-AD mice treated with water only.

### DETAILED DESCRIPTION OF THE INVENTION

The present invention has been described in terms of one or more preferred embodiments, and it should be appreciated that many equivalents, alternatives, variations, and modifications, aside from those expressly stated, are possible and within the scope of the invention.

The present invention relates to methods of treatment and formulations for treatment of Alzheimer's disease and dementia associated with Down Syndrome. The inventors have surprisingly found that Alzheimer's disease can be treated by the administration of DFMO at dosages that are both efficacious and demonstrate a low toxicity profile′ where adverse events are limited to Grade 1 or no adverse events are found. Grade 1 adverse events are defined as “mild; asymptomatic or mild symptoms, clinical or diagnostic observations only; intervention not indicated” in accordance with the common toxicity criteria for adverse events (CTCAE; Version 4 CTCAE; Version 4, May 28, 2009, U.S. DEPARTMENT OF HEALTH AND HUMAN SERVICES National Institutes of Health). The unexpected potency of DFMO to treat Alzheimer's disease has been shown in a mouse model of Alzheimer's disease at dosages with a low toxicity profile and are currently lower than dosages that have been used for the treatment of other diseases.

In one embodiment, the disclosure provides a method of reducing, inhibiting or postponing one or more symptom of Alzheimer's disease or dementia associated with Down Syndrome in a patient in need thereof, comprising, consisting essentially of, or consists of administering a therapeutically effective amount of a pharmaceutical composition comprising, consisting essentially of, or consists of a therapeutically effective amount of an ornithine decarboxylase inhibitor or a pharmaceutically acceptable salt thereof. In other embodiments, the present disclosure provides a method of treating Alzheimer's disease or dementia associated with Down Syndrome in a subject in need thereof, the method comprising, consisting essentially of, or consisting of orally administering said subject a therapeutically effective amount of an ornithine decarboxylase inhibitor or a pharmaceutically acceptable salt thereof.

In a preferred embodiment, the ornithine decarboxylase inhibitor is alpha-difluoromethylornithine (DFMO).

By “treating” or “treatment” we mean the management and care of a subject for the purpose of combating and reducing the disease, condition, or disorder. The terms embrace preventative, i.e., prophylactic, and palliative treatments. Treating includes the administration of an ornithine decarboxylase inhibitor, preferably alpha-difluoromethylornithine of the present invention to reduce, inhibit, ameliorate and/or improve the onset of the symptoms or complications, or alleviating the symptoms, complications of the Alzheimer's disease or dementia associated with Down Syndrome, diminishment of or delay in the appearance of or worsening of any direct or indirect pathological consequences of the disease, decrease of the rate of disease progression and amelioration or palliation of the disease state. Desirable effects of the treatment may result in the reduction, inhibition, suppression or amelioration of one or more symptoms of Alzheimer's disease. As used herein, the term “treatment” is not necessarily meant to imply cure or complete abolition of the Alzheimer's disease. Treatment may refer to the inhibiting or slowing of the progression of the onset of Alzheimer's disease, reducing the incidence of Alzheimer's disease, or preventing further cognitive degradation.

By “ameliorate,” “amelioration,” “improvement” or the like, we mean a detectable improvement or a detectable change consistent with improvement occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values. Such improvement or change may be observed in treated subjects as compared to subjects not treated with the formulations of the present invention, where the untreated subjects have, or are subject to developing, the same or similar disease, condition, symptom or the like. Amelioration of a disease, condition, symptom or assay parameter may be determined subjectively or objectively, e.g., self-assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of cell migration within a subject. Amelioration may be transient, prolonged or permanent, or it may be variable at relevant times during or after the formulation of the present invention is administered to a subject.

By “reduce,” “inhibit,” or the like, we mean a detectable reduction or a detectable change consistent with reduced neurological symptom and/or improved mental control occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range about between any two of these values.

“Pharmaceutically acceptable” as used herein means that the compound or composition is suitable for administration to a subject to achieve the treatments described herein, without unduly deleterious side effects in light of the severity of the disease and necessity of the treatment.

Alzheimer's disease (AD) is a progressive neurodegenerative disorder characterized by progressive cognitive impairment (loss of memory, cognition and behavioral instability) due to neuronal loss and resulting in language disorders, problems with judgement, problem solving, planning, abstract thought, apraxia, deficits in visual functions and dementia. AD diagnosis is made primarily using cognitive assessments and secondarily can be coupled with brain volume changes using MRI compared to some standard control individual or over time in the same individual. Cognitive tests used clinically as a measure of a reduction of the deterioration include, but are not limited to, mini-mental state examination (MMSE) or Alzheimer's Disease Assessment Scale-Cognitive Subscale (ADAS-Cog) test. Alternative methods of determining AD, include, in some cases, volume measurement of the brain to detect the stopping of AD brain volume loss usually associated with AD using magnetic resonance imaging MRI.

“Chronic administration” as used herein refers to administration over a prolonged period of time, as opposed to acute administration in which administration is terminated when a disease is sufficiently treated or cured. For example, chronic administration may be administration multiple times a week (e.g., one, two or three times daily for three, four, five, six, or seven days a week) for a period of at least one, two, three, four, five or six months or more, after which time disease progression is reassessed by the treating physician and dosage is continued or adjusted as necessary, with treatment typically continuing on an ongoing basis. In some embodiments, the pharmaceutical compositions or ornithine decarboxylase inhibitors are administered for at least 6 months, alternatively at least 12 months, alternatively at least 24 months, alternatively at least 36 months, alternatively at least 36 months, alternatively at least 48 months, alternatively at least 60 months, alternatively at least 72 months, alternatively at least 84 months, alternatively at least 96 months, alternatively at least 108 months, and any amount or range in between (e.g. 7 months, 8 months, 9 months, 10 months, etc.).

In some embodiments, the chronic administration may be on an interrupted dosing schedule. For example, treatment may be given weekly, biweekly, every other day, every third day, or other configurations, including, for example, once a week, twice a week, three times a week, four time a week, five times a week, every other day, every third day, every fourth day, etc.

The present invention is primarily concerned with the treatment of human subjects, but the invention may also be carried out on animal subjects, particularly mammalian subjects such as mice, rats, dogs, cats, livestock and horses for veterinary purposes, and for drug screening, testing and development purposes. In some embodiments, the present invention is directed to the treatment of a human with Alzheimer's disease. In other embodiments, the present inventions is directed to the treatment of a human with dementia associated with Downs Syndrome.

The disclosures of all United States Patent references cited herein and non-patent literature are incorporated by reference herein in their entirety.

### 1. Active Compounds.

Ornithine decarboxylase inhibitors are known and described in, for example, U.S. Pat. Nos. 5,753,714; 5,132,293; 5,002,879; 4,720,489; and 4,499,072. Examples include, but are not limited to, alphadifluoromethylornithine, 2-(difluoromethyl)-2,5-diaminopentanoic acid; alpha-ethynyl ornithine; 6-heptyne-2,5-diamine; 2-methyl-6-heptyne diamine; alpha.-difluoromethyl ornithine; the methyl ester of monofluoromethyl dehydroornithine; the R,R-isomer of methyl acetylenic putrescine, 3-aminooxy-1-aminopropane (APA) and its analogs or derivatives such as CGP 52622A and CGP 54169A, 1,25-dihydroxycholecalciferol, and pharmaceutically acceptable salts and prodrugs thereof. The methods provided herein can also be used to treat dementia associated with Down Syndrome. Dementia associated with Down Syndrome shares similar pathology with Alzheimer's disease. Down syndrome is a developmental disorder involving triplication of chromosome 21 and is a common cause of genetically based intellectual disability. Individuals with Down syndrome show abnormal memory and learning processes beginning at an early age and worsening by the fifth decade of life (Wisneiwski 1985). Pathology consisting of amyloid plaques, hyperphosphorylated tau protein, neuron loss, metabolite changes detected by magnetic resonance spectroscopy and increased neuroinflammation are observed in brains of Down syndrome individuals at later stages of life (Lin A L, 2016). These pathologies are consistent with an Alzheimer's disease pathology.

Additional examples of ornithine decarboxylase inhibitors that may be used to carry out the present invention include but are not limited to: TABLE-US-00001 Drug Company CGP-52622A Novartis AG CGP-54169A Novartis AG dihydroxycholecalciferol Chugai Pharmaceutical Co Ltd insulin analogs, Scios/Pfizer Scios Inc CGP-51905A Novartis AG CGP-45300A Novartis AG anticancer agents, University of University of Illinois vitamin D analogs (cancer), Johns Hopkins University Cytochroma/Johns Hopkins.

In a preferred embodiment, the ornithine decarboxylase inhibitor also serves as a partial antagonist of arginase. A preferred inhibitor, alpha-difluoromethylornithine (DFMO; eflornithine (sometimes called “elfornithine”), is known and described in, for example, U.S. Pat. Nos. 6,730,809; 6,573,290; 6,258,845; and 4,925,835.

The active compounds disclosed herein can, as noted above, be prepared in the form of their pharmaceutically acceptable salts.

Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

### 2. Formulations and Administration.

The active compounds described above may be formulated for administration in a pharmaceutical carrier in accordance with known techniques. See, e.g., Remington, The Science and Practice of Pharmacy (9th Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well-known techniques of pharmacy comprising admixing the components, optionally including one or more accessory ingredients.

The formulations of the invention include those suitable for oral administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder. Alternatively, the formulation may comprise a liquid formulation. In some embodiments, the liquid formulation may be added to a beverage, for example a juice.

Capsules may contain particles coated with the active agents. Particles used herein may be of any suitable size, typically from about 100 to 1000 micrometers in diameter. Examples include particles with a diameter of about 600 to 250 urn (30-60 mesh), or a diameter of 700 to 600 um (25-30 mesh). Size of particles can be determined in accordance with known techniques, such as described in the CRC Handbook, 64th edition, page F-114 and USP24/NF19, page 1969. Any suitable core material can be used for the particles. Examples of such materials are polymers e.g., plastic resins; inorganic substances, e.g., silica, glass, hydroxyapatite, salts (sodium or potassium chloride, calcium or magnesium carbonate) and the like; organic substances, e.g., activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like saccharides. In addition, the particles according to the present invention may further contain one or more additional additives such as thickening agents, lubricants, surfactants, preservatives, complexing and chelating agents, electrolytes or other active ingredients, e.g., antiinflammatory agents, antibacterials, disinfectants or vitamins. The particles may be inserted into a suitable capsule such as a gelatin capsule in accordance with known techniques.

Tablets can be produced by conventional tabletting techniques with conventional ingredients or excipients. The tablets are preferably formed from a composition comprising the particles described herein distributed in a mixture of a disintegrant and a diluent or filler. Suitable disintegrants include, but are not limited to, crospovidone and croscarmellose. Suitable diluents include, but are not limited to, lactose, sucrose, dextrose, mannitol, sorbitol, starch, cellulose, calcium phosphate, microcrystalline cellulose such as AVICEL™, etc. Tablets may include a variety of other conventional ingredients, such as binders, buffering agents, lubricants, glidants, thickening agents, sweetening agents, flavors, and pigments.

Liquids can be prepared in any suitable form, such as a syrup, in accordance with known techniques by dissolving, solubilizing or suspending (e.g., as an emulsion or microemulsion) the active agents therein. Such liquids may be aqueous liquids, optionally including additional cosolvents such as oils and the like.

The therapeutically effective dosage of the ornithine decarboxylase inhibitors, the use of which is in the scope of present invention, will vary somewhat from compound to compound, and patient to patient, and will depend upon factors such as the age and condition of the patient and the route of delivery. Dosages are within the range of a low toxicity profile′ defined as the restriction of adverse events to Grade 1 or to no adverse events with DFMO treatment. Adverse event is commonly defined as any untoward medical occurrence in a patient administered a pharmaceutical product and which does not necessarily have a causal relationship with the treatment. Grade 1 adverse events are defined as “mild; asymptomatic or mild symptoms, clinical or diagnostic observations only; intervention not indicated” in accordance with the common toxicity criteria for adverse events (CTCAE; Version 4, May 28, 2009, U.S. DEPARTMENT OF HEALTH AND HUMAN SERVICES National Institutes of Health) that defines the severity of adverse events.

An oral dose formulation for treating Alzheimer's disease or dementia associated with Down Syndrome comprises, consisting essentially of or consists of a dose of an ornithine decarboxylase inhibitor or a pharmaceutically acceptable salt thereof in an amount effective to treat Alzheimer's disease that is both efficacious and demonstrates a low toxicity profile′ where adverse events are limited to Grade 1 or no adverse events are found. Grade 1 adverse events are defined as “mild; asymptomatic or mild symptoms, clinical or diagnostic observations only; intervention not indicated” in accordance with the common toxicity criteria for adverse events (CTCAE; Version 4 CTCAE; Version 4, May 28, 2009, U.S. DEPARTMENT OF HEALTH AND HUMAN SERVICES National Institutes of Health)

The surprising potency of DFMO in the treatment of AD-like symptoms in a mouse model of AD limits toxicity to no adverse events in the treated mice. This is an extra-added benefit of DFMO as a therapeutic since it has been shown for other diseases that higher dosages of DFMO leads to unwanted and undesirable adverse events ranging from Grade 2 to Grade 5 (as defined by CTCAE; Version 4 May 28, 2009, U.S. DEPARTMENT OF HEALTH AND HUMAN SERVICES National Institutes of Health), that may reduce the likelihood of a patient continuing treatment. AD is a slowly developing and long duration disease of aging humans that requires long term treatment with a drug that shows no adverse events or adverse advents no greater that Grade 1 while maintaining high therapeutic efficacy. The toxicity of DFMO at higher levels has been shown in a number of clinical trials. For example, in human clinical trials on individuals with various forms of cancers (TABLE 4), DFMO shows dose limiting cytopenia at 2000 mg/m²/d (IV) (Lipton A.) where dose limiting is defined as treatment that is serious enough to prevent an increase in dose or level of that treatment [Paoletti X.). This represents a 22 fold to 2200 fold higher dose than that required for reasonable efficacy (0.9 to 90 mg/m²/d oral delivery) in a mouse model of AD. DFMO shows a dose limiting diarrhea in humans at 6000 mg/m²/d oral delivery (Meyskens F L). This dose is a 67 fold to 6700 fold higher dose than that required for reasonable efficacy in a mouse model of AD. DFMO shows a dose limiting ototoxicity at 1500 mg/m²/d oral delivery and mild reversible ototoxicity (no adverse event grade assigned) at 1000 mg/m²/d oral delivery [Lipton A] [Pasic T R]. These doses correspond to 11 to 1100 and to 17 to 1700 fold higher dose of DFMO than that required for reasonable efficacy in a mouse model of AD. In a different study, mild ototoxicity was found at a DFMO dose of 2000 mg/m²/d by oral delivery (22 to 2200 fold higher) [Croghan M K] suggesting variability in the dose limiting effects on hearing loss with DFMO. Given the published variability in ototoxicity thresholds, doses from 1000 to 1500 mg/m²/d by oral delivery is considered to serve as dose limiting. In the preferred embodiments, the alpha-difluoromethylornithine is the only active ingredient being administered to the patient

In some embodiments, the alpha-difluoromethylornithine (DFMO) is included in the formulation or administered to the subject in an amount effective to deliver a dosage thereof of about 10 milligrams per meter² to about 400 milligrams per meter² per day to the subject. In one embodiment, DFMO is included in the formulation or administered to the subject at a dosage of about 0.9 to about 90 mg/m²/day. Suitably, the formulation the therapeutically effective amount of DFMO is a dosage of 1 to 100 milligrams per m²; alternatively a dosage of 101 to 200 milligrams per m²; alternatively a dosage of 201 to 300 milligrams per m²; alternatively a dosage of 301 to 400 milligrams per m².

Dosages higher than 400 milligrams per meter² are contemplated, although the higher values increase the likelihood of Grade 1, 2 or greater adverse events. In an embodiment, the alpha-difluoromethylornithine is administered with an additional AD treatment to the patient. Suitable AD treatments include, but are not limited to, for example, Thalidomide, Donepezil, Tacrine, Revastigmine, Memantine, Galantamine, Rosiglutizone, Pioglutazone, Sulindac, gmCSF, anti Abeta antibodies, anti-tau antibodies, gamma secretase inhibitors, beta secretase inhibitors, extended use version of the above, or drugs formulated as combinations of the above.

The following non-limiting examples are included for purposes of illustration only, and are not intended to limit the scope of the range of techniques and protocols in which the compositions and methods of the present invention may find utility, as will be appreciated by one of skill in the art and can be readily implemented.

### EXAMPLES

### Example 1: Treatment of Alzheimer's Disease with DFMO

Alzheimer's disease (CVN-AD) mice are modified non-human warm-blooded vertebrate animals in which a biologically active human APP polypeptide is expressed, and in which function of its inducible Nitric Oxide Synthase (iNOS) protein is reduced as compared to a non-modified animal. CVN-AD mice are described in U.S. Patent Publication No. 20090081128, the contents of which are incorporated by reference in their entirety.

CVN-AD mice show pathology that includes (1) expression of mutated human APP at approximately 0.8-fold of the normal mouse APP level (Miao, J); (2) Aβ deposition around blood vessels resembling pathology observed in humans with cerebral amyloid angiopathy (CAA) (Attems, J.); (3) hyperphosphorylated and aggregated mouse tau; neuronal loss; worsening behavioral deficits with time (Colton, C.); and (4) significant hippocampal and fornix volume loss (Badea, A.).

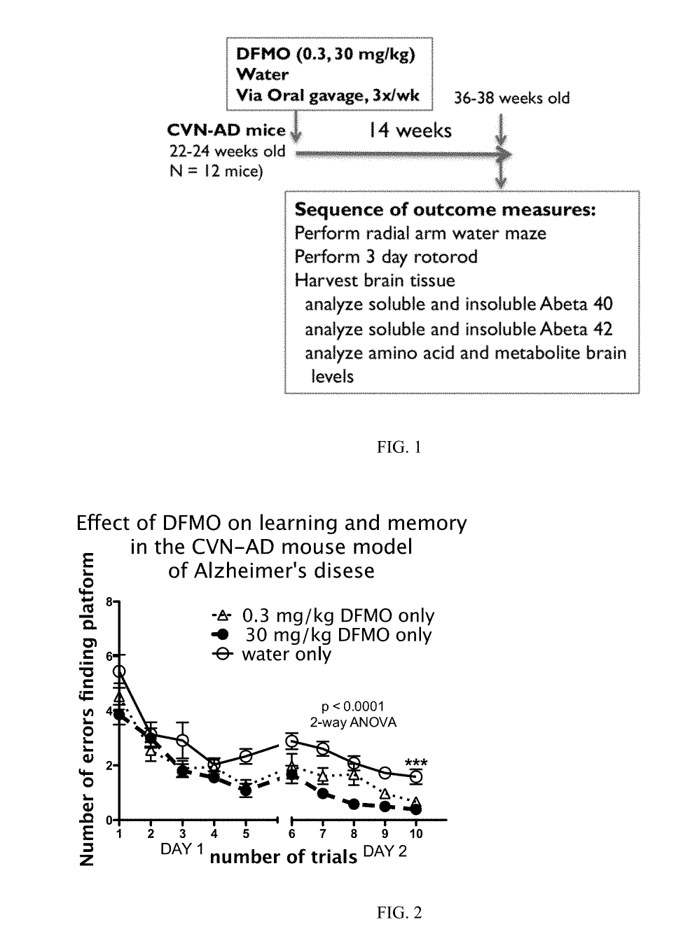

This example was carried out as schematically illustrated in FIG. 1. Briefly, CVN-AD mice (n=12 per group) were administered DFMO (either 0.3 mg/kg or 30 mg/kg) or water (vehicle) by oral gavage (3×/wk) for 14 weeks starting at 22 to 24 weeks of age. Control (water treated) mice were provided the same food and water but without added DFMO, and kept under the same conditions as the DFMO-treated mice. DFMO treated and water treated (control) mice were subjected in the same randomized manner to the 2-day radial arm water maze, followed by the 3-day rotorod. Brains from each mouse were harvested at the end the third day of the rotorod procedure. Brain tissue lysates from each mouse in the experiment were used to determine the levels of Abeta 40 and Abeta 42 peptides and brain metabolites.

As shown in FIG. 2 the average number of errors (plus and minus the standard error of the mean) in finding an escape platform after 2 days of trials in a standard radial arm water maze is statistically significantly decreased by treatment with 0.3 mg/kg (0.9 mg/m²) and more so with 30 mg/kg (92.5 mg/m²) when compared to Alzheimer's disease mice treated in the same manner with water only. This age in the CVN-AD mouse model is considered as representative of early Alzheimer's disease in humans (Colton, C.). The number of mice in each group is 12.

As shown in FIG. 3, the average levels of insoluble Abeta 42 peptide and insoluble Abeta40 peptide (plus and minus the standard error of the mean) measured in whole brain lysates are shown in FIG. 3A. No significant change was found with DFMO treatment. FIG. 3B shows the dose-dependent effect of difluoromethylornithine (DFMO) on soluble Abeta 42 peptide and soluble Abeta 40 peptide (plus and minus the standard error of the mean) in a mouse model of human Alzheimer's disease. The average levels of soluble Abeta 42 peptide and soluble Abeta40 peptide (plus and minus the standard error of the mean) measured in whole brain lysates are shown in FIG. 3B. Soluble Abeta 40 levels were statistically significantly decreased with DFMO treatment. ** p<0.01; * p<0.05. The number of mice for each group was 12. Brain tissue data are taken from the same mice as shown in FIG. 2.

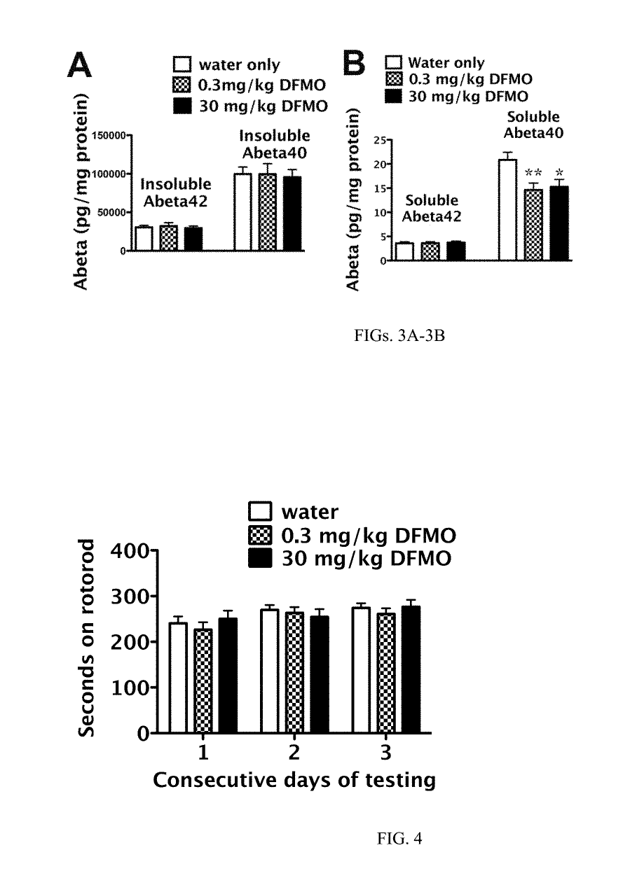

FIG. 4 shows that treatment of CVN-AD mice with DFMO does not alter motor skills or co-ordination as tested by the 3-day rotorod behavioral test compared to CVN-AD mice treated with water only. No change in the average number of seconds on the rotorod were observed over 3 days of testing for the same groups of mice shown in FIGS. 2 and 3. DFMO-treatment at the doses used resulted in efficacy against cognitive deficits and Abeta accumulation, but with no toxic effect on motor skills, an additional brain function.

Table 1 shows that DFMO treatment of CVN-AD mice compared to CVN-AD mice treated with water only results in altered whole brain lysate metabolite levels that are directly related to ornithine decarboxylase activity in the brain. DFMO treatment resulted in significantly lower brain levels of putrescine and significantly lower brain levels of metabolites (spermidine, spermine) in a mouse model of human Alzheimer's disease compared to CVN-AD mice that remained untreated. Humans with Alzheimer's disease show elevation of these metabolites (Inoue K). DFMO restores brain metabolites in the CVN-AD mouse model of AD by reducing the levels observed in the disease state.

TABLE 1

| | | CVN-AD | |
|---|---|---|---|
| | | brain lysate | |
| | | levels | |
| | CVN-AD | DFMO- | |
| | brain lysate | treated | |
| Metabolite | levels | (30 mg/kg) | |
| directly | Water only | (Average | |
| related to | (Average uM/g +/− | uM/g +/− | |
| DFMO | sem; n = | sem; n = | Observed |
| activity | 11-12 mice) | 11-12 mice) | difference |
| putrescine | 0.69 +/− 0.08 | 0.49 +/− 0.02 | p < 0.05 |
| | | | Significantly |
| | | | decreased |
| | | | with DFMO- |
| | | | treatment |
| spermidine | 16.2 +/− 1.16 | 12.2 +/− 0.63 | p < 0.01 |
| | | | Significantly |
| | | | decreased |
| | | | with DFMO- |
| | | | treatment |

Table 1 shows that DFMO treatment of CVN-AD mice compared to CVN-AD mice treated with water only results in altered whole brain lysate metabolite levels that are directly related to ornithine decarboxylase activity in the brain.

### Example 2: Determination of Dosages with Low Toxicity and Efficacy; Dosages for DFMO for Alzheimer's Disease Trial

We compared historical data from the literature on dosages of DFMO in human trials and the data from our pre-clinical studies using the mouse model of Alzheimer's disease, (CVN-AD mice) to estimate dosages for human clinical trials for the treatment of Alzheimer's disease.

### Table 2: Trial Summary of DFMO in CVN-AD Mice

TABLE 2
Trial Summary of DFMO in CVN-AD mice

| | | Dose | On target in |
|---|---|---|---|
| | Length of | limiting | brain (see |
| Mouse | trial | events* | data figures) |
| 0.3 mg/kg | 14 wks | none | yes |
| 10 mg/kg | 14 wks | none | yes (data not |
| | | | shown) |
| 30 mg/kg | 14 wks | none | yes |

*defined as: >15% body weight loss; self imposed hiding/isolation; altered breathing patterns; increased or decreased movement; signs of lose stool; unkempt appearance; abnormal posture; muscle rigidity or lack of muscle tone; twitching, tremor or trembling; increased salivation. Guidelines for Pain and Distress Signs in Mice, Duke University Animal Care and Use Program. 2016

The use of DFMO as a therapy for Alzheimer's disease is unanticipated based on previously published findings in the field. As an example, brain infections in humans caused by Trypanosoma brucei gambiense (referred to herein as trypanosomiasis) are successfully treated with DFMO. The effective dosage for successful treatment is 14800 milligrams per meter-squared per day (14800 mg/m2/d) given either orally (by mouth) or 14800 milligrams per meter-squared per day (14800 mg/m2/d) administered by an intravenous route (IV) ((Na-Bangchang, K.) (Priotto, G.). (Table 3).

TABLE 3
EFFICACY AND TOXICITY DATA Treatment of Brain
trypanosomiasis with DFMO (no adverse event scale used)
DFMO-associated toxicity

| | | Brain - | | | |
|---|---|---|---|---|---|
| | | based | | Other Toxicity | |
| Daily dose g/m²/d | Efficacy | toxicity | Cytopenia | detected | reference |
| 14.8 g/m²/d | Partial clearance of | Not | 66% | Headache | (Na-Bangchang, K) |
| (400 mg/kg/day) | brain trypanosomes; | determined | | (16.7%), | |
| ORAL (n = 25 | Reduced | | | diarrhea | |
| subjects) | signs/symptoms of | | | (58.3%) | |
| | trypansosmiasis | | | | |
| | (headache, fever, | | | | |
| | tremor, chorea, | | | | |
| | inability to walk, | | | | |
| | inactivity, general | | | | |
| | weakness). No | | | | |
| | death | | | | |
| 14. g/m²/d | Prolonged clearance | Seizures | 21.6% | Abdominal | (Priotto, G.) |
| (400 mg/kg/day) IV | of trypanosomes; | (4%) | | pain (37.7%), | |
| (n = 1055 subjects) | 44% confirmed | 15 deaths | | headache | |
| | cured, 45% | | | (31.2%), | |
| | probable cure | | | diarrhea | |

The efficacious dosage in brain trypanosomaisis demonstrates characteristics resembling Grade 2, 3, 4 and 5 adverse events as defined by CTCAE; (Version 4 May 28, 2009, U.S. DEPARTMENT OF HEALTH AND HUMAN SERVICES National Institutes of Health) and include headache, diarrhea, abdominal pain, cytopenia, seizures and death (Table 3). In contrast, treatment of the APPSwDI/mNos2−/− mouse (abbreviated CVN-AD) model of Alzheimer's Disease and as shown in FIGS. 2 and 3 and Table 2 demonstrates that DFMO has efficacy against Alzheimer's-like neurodegenerative pathology at a range of doses including those from 0.9 to 90 mg/m2/d (Table 2).

No observable adverse events (as defined in Table 2) are shown in the dose range of 0.9 to 90 mg/m2/d in DFMO treated CVN-AD mice that model Alzheimer's disease; confirming the lack of dose-limiting events compared to those dose limiting events such as death, seizures, or diarrhea as observed in humans treated with efficacious doses of DFMO for trypanosomiasis (Na-Bangchang K.). The differences in the efficacious doses show the difference between the pharmaceutical potency found in a mouse model of AD at DFMO drug concentrations ranging from 0.9 to 90 mg/m2/d and those found in human typranosomiasis at a DFMO drug concentration of 14800 mg/m2/d. Using these numbers, we calculate an unforeseen increase in the potency with which DFMO can treat AD as ranging from 164 fold to 16444 fold greater than DFMO's ability to treat trypanosomiasis.

TABLE 4
General TOXICITY DATA for cancer-based treatment with DFMO

| Daily dose | | Brain - based | | Other Toxicity | |
|---|---|---|---|---|---|
| g/m^{2/d} | Target | toxicity | Cytopenia | detected | reference |
| 0.5-3 g/m²/d | Normal | Ototoxicity (0.5 g/m²/d | Not | Vomiting/diarrhea | (Pasic, TR) |
| (oral) (n = 13 | volunteers | (9%) same as placebo | determined | Not study limiting | |
| subjects) | | (12%); >1 g/m²/d is | | | |
| | | dose dependent, | | | |
| | | increasing toxicity | | | |
| 2 g/m²/d (oral) | melanoma | Ototoxicity (10%) | Not | Not determined | (Croghan, MK) |
| (n = 16 subjects); | | Increasing with dose | determined | | |
| 75 days | | and duration | | | |
| 6 g/m²/d (oral) | melanoma | Ototoxicity in 50% of | 1/24 | 67% (low grade) | (Meyskens FL) |
| escalated to12 g/m²/d | | treated subjects | | | |
| (n = 24 | | (12/24) | | | |
| subjects) | | | | | |
| 1 g/m²/d (Intra | Metastatic | Ototoxicity (0/3) at 0.5 | (0/3) at 0.5 | 0/3) at 0.5 and 1 g/m²/d | (Lipton, A.) |
| Venous-IV) | liver | and 1 g/m²/d (IV | and 1 g/m²/d | (IV) | |
| translates to | | dose) | (IV) | (0/3) at 2 g/m²/d | |
| 1.5 g/m²/d oral | | Ototoxicity (3/3) at 2 g/m²/d | (2/3) at 2 g/m²/d | (IV) | |
| (n = 3 × 3 | | (IV) | (IV) | | |
| escalating dose) | | | | | |

Table 4 shows a summary from historic literature of doses of DFMO associated with dose limiting effects, including ototoxicity, cytopenia and gastrointestinal upset. Dose limiting is defined as treatment that is serious enough to prevent an increase in dose or level of that treatment [Paoletti X.). Specifically, higher dosages of DFMO (>1500 mg/m²) are typically associated with dose limiting effects.

Table 5 demonstrates a calculation for suitable dosages and estimated therapeutic indices which are calculated based on the results of the mouse model of AD and which can be used in human trial for Alzheimer's disease.

TABLE 5
Dose range translation from available mouse data to Human Usage

| | | | | | Calculated | |
|---|---|---|---|---|---|---|
| | | | | | Therapeutic Index | |
| | | | | | USING | USING |
| | | | HUMAN | HUMAN | 1000 mg/M² | 1500 mg/M² |
| | MOUSE | | EQUIVALENT | EQUIVALENT | as the | As the |
| MOUSE | DOSE | Approx. | mg/kg | mg/M² | Limiting | Limiting |
| DOSE | (mg/M² × | micromol | (=MOUSE | (=HUMAN | Toxic | Toxic |
| mg/kg | 3) | in brain | mg/kg × 1/12) | mg/kg × 37) | Dose | Dose |
| 0.3 | 0.9 | 0.3 | 0.025 | 0.925 | 1075 | 1612 |
| 10 | 30 | 1 | .83 | 30.7 | 33 | 49 |
| 30 | 90 | 3 | 2.5 | 92.5 | 11 | 16 |
| 100 | 300 | 10 | 8.3 | 307 | 3.3 | 4.9 |

Table 6 shows the estimated dosages to be used in the human trials. As demonstrated by Table 5 and 6, the dosages of DFMO to be used in humans for treatment of Alzheimer's disease are significantly lower than dose-limiting dosages used for treatment of the brain disease, trypanosomiasis.

TABLE 6
Predicted dosage for Alzheimer's Clinical Trial

| Estimated Human | | | Predicted Dose |
|---|---|---|---|
| Dose | Frequency | Duration | limiting effects |
| placebo | daily | 6 mos-4 yrs | none |
| 36 mg/m² | daily | 6 mos-4 yrs | none |
| 109 mg/m² | daily | 6 mos-4 yrs | none |
| 328 mg/m² | daily | 6 mos-4 yrs | none |

### REFERENCES

Castellani, R. J.; Perry, G. Arch Med Res., 43, 694-698, 2012 Pathogenesis and disease modifying therapy in Alzheimer's disease: the flat line of progress.
Miao J; Xu F., Davis, J. Otte-Holler, I., Verbeek, M. Van Nostrand, W E, Am. J. Pathol. 167, 505-515, 2005 Cerebral microvascular amyloid beta protein deposition induces vascular degeneration and neuro-inflammation in transgenic mice expressing human vasculotropic mutant amyloid beta precursor protein;
Badea, A; Kane, L.; Anderson, R. J.; Qi, Y., Foster, M.; Cofer, G.; Medvitz, N.; Buckley, A. F.; Badea, A. K.; Wetzel, W., Colton C A, Neuroimage, Aug. 16, 2016 The fornix provides multiple biomarkers to characterize circuit disruption in a mouse model of Alzheimer's disease.
Attems, J.; Lintner, F.; Jellinger, K. A. Acta Neuropathol, 107 283-91, 2004 Amyloid beta peptide 1-42 highly correlates with capillary cerebral amyloid angiopathy and Alzheimer disease pathology
Colton, C. A.; Wilson, J. G.; Everhart, A; Wilcock, D. M.; Puolivali, J.; Heikkinen, T.; Oksman, J.; Jaaskelainen, O.; Lehtimaki, K.; Laitinen, T.; Vartiainen, N.; Vitek, M. P. J Neuropathol Exp Neurol; 73; 752-769; 2014; mNos2 Deletion and Human NOS2 Replacement in Alzheimer Disease Models
Inoue, K., Tsutsui, H.; Akatsu, H.; Hashizume, Y.; Matsukawa, N.; Yamamoto, T Toyo'oka, T. Sci Rep, 3, 2364-, 2013 Metabolic profiling of Alzheimer's disease brains
Na-Bangchang, K., Doua, F., Konsil, J., Hanpitakpong, W., Kamanikom, B., Kuzoe, F., Eur. J. Clin. Pharmacol. 60, 269-278, 2004. The pharmacokinetics of eflornithine (alpha-difluoromethylornithine) in patients with late-stage T.b. gambiense sleeping sickness.
Lipton, A.; Harvey, H. A.; Glenn, J.; Weidner, W. A.; Strauss, M.; Miller, S. E.; Taylor, J. B.; White-Hershey, D.; Barlow, J. L. Cancer 63 433-437, 1989 A phase 1 study of hepatic arterial infusion using difluoromethylornithine.
Pasic, T. R.; Heisey, D.; Love, R. R.; Arch Otolaryngol Head Neck Surg 123 1281-1286, 1997 alpha-difluoromethylornithine Chemopreventaion clinical trial results
Croghan, M. K.; Dose-related alpha difluoromethylornithine ototoxicity, Am J Clin Oncol 14 331-335 1991.
Priotto, G.; Pinoges, L.; Fursa, I.; Burke, B.; Nicolay, N.; Grillet, G.; Hewison, C.; Balasegaram, M. British Med. J 336 705-708 2008 Safety and effectiveness of first line eflornithine for Trypanosoma brucei gambiense sleeping sickness in Sundan: cohort study.
Paoletti, X.; LeTourneau, C.: Verweij, J.; Siu, L. L.; Seymour, L.; Postel-Vinay, S.; Collette, L.; Rizzo, E.; Ivy, P.; Olmos, D.; Massard, C.; Lacombe, D., Kaye, S. B.; Soria J. C., Eur J Cancer 50 2050-2056, 2014.
Meyskens, F. L.; Kingsley, E. M.; Glattke, T.; Loescher, L.; Booth, A. Invest. New Drugs 4 257-262, 1986. A phase II study of alpha-difluoromethylornithine (DFMO) for the treatment of metastatic melanoma.
Qu, N.; Ignatenko, N. A.; Yamauchi, P.; Stringer, D. E.; Levenson, C.; Shannon, P.; Perrin, S.; Gerner, E. W., Biochem J 375 465-470, 2003. Inhibition of human ornithine decarboxylase activity by enantiomers of difluoromethylornthine.
Coleman, S. C.; Stanley, B. A.; Pegg, A. E., J Biol Chem 268 24572-24579, 1993. Effects of mutations at active site residues on the activity of ornithine decarboxylase and its inhibition by active site-directed irreversible inhibitors.
Priotto, G.; Bastard, M.; Flevaud, L.; Etard, J. F. Early prediction of treatment efficacy in second stage gambiense human African trypanosomiasis Br Med. J. 336:705 2006.

1. A method of treating Alzheimer's disease in a subject in need thereof, the method comprising orally administering said subject a therapeutically effective amount of an ornithine decarboxylase inhibitor or a pharmaceutically acceptable salt thereof.

2. The method of claim 1, wherein said subject is a human.

3. The method of claim 1, wherein said ornithine decarboxylase inhibitor is alpha-difluoromethylornithine or a pharmaceutically acceptable salt thereof.

4. The method of claim 1, wherein said administering steps is performed chronically for at least six months.

5. The method of claim 1, wherein said administering steps is performed chronically for at least twelve months.

6. The method of claim 1, wherein said administering steps is performed chronically for at least eighteen months.

7. The method of claim 1, wherein said administering step is performed for at least 36 months.

8. The method of claim 1, wherein said administering steps is performed chronically for at least 48 months.

9. The method of claim 1, wherein the administering step is a chronic administering step.

10. An oral dose formulation for treating Alzheimer's disease comprising a dose of an ornithine decarboxylase inhibitor or a pharmaceutically acceptable salt thereof in an amount effective to treat Alzheimer's disease that is both efficacious and demonstrates a low toxicity profile with no adverse effects or adverse effects of Grade 1 or lower.

11. The oral dose formulation of claim 10, wherein the ornithine decarboxylase inhibitor is alpha difluoromethylornithine or a pharmaceutically acceptable salt thereof.

12. The formulation of claim 10, wherein said ornithine decarboxylase inhibitor is alpha difluoromethylornithine, or a pharmaceutically acceptable salt thereof, is included in said formulation in an amount effective to deliver a dosage of 1 to 100 milligrams per m²; 101 to 200 milligrams per m²; 201 to 300 milligrams per m²; or 301 to 400 milligrams per m².

13. The formulation of claim 11, wherein the formulation delivers a dosage of about 100 mg/m² to about 400 mg/m² per day.

14. The formulation of claim 11, wherein the formulation is in the form of a tablet.

15. The formulation of claim 11, wherein the formulation is in the form of a capsule.

16. The formulation of claim 11, wherein the formulation is in the form of a liquid.

17. The formulation of claim 11, wherein ornithine decarboxylase inhibitor is the only active ingredient in the formulation.

18. The formulation of claim 10, consisting essentially of an ornithine decarboxylase inhibitor and a pharmaceutically acceptable carrier.

19. The formulation of claim 18, wherein the acceptable carrier is water or fruit juice.

20. A method of treating dementia associated with Down Syndrome in a subject in need thereof, the method comprising orally administering said subject a therapeutically effective amount of an ornithine decarboxylase inhibitor or a pharmaceutically acceptable salt thereof.

21. The method of claim 20, wherein said subject is a human.

22. The method of claim 20, wherein said ornithine decarboxylase inhibitor is alpha-difluoromethylornithine or a pharmaceutically acceptable salt thereof.

23. The method of claim 20, wherein said administering steps is performed chronically for at least six months.

24. The method of claim 20, wherein said administering steps is performed chronically for at least twelve months.

25. The method of claim 20, wherein said administering steps is performed chronically for at least eighteen months.

26. The method of claim 20, wherein said administering step is performed for at least 36 months.

27. The method of claim 20, wherein said administering steps is performed chronically for at least 48 months.

28. The method of claim 20, wherein the administering step is a chronic administering step.

29. The method of claim 20, wherein the subject is a human with Down Syndrome.

30. An oral dose formulation for treating dementia associated with Down Syndrome comprising a dose of an ornithine decarboxylase inhibitor or a pharmaceutically acceptable salt thereof in an amount effective to treat dementia associated with Down Syndrome that is both efficacious and demonstrates a low toxicity profile with no adverse effects or adverse effects of Grade 1 or lower.
